(19) United States
(12) Patent Application Publication
(10) Pub. No.: US 2015/0282739 A1
(43) Pub. Date: Oct. 8, 2015

(54) BIOLOGICAL INFORMATION ACQUISITION APPARATUS AND BIOLOGICAL INFORMATION ACQUISITION METHOD

(71) Applicant: SEIKO EPSON CORPORATION, Tokyo (JP)

(72) Inventors: Kazuhiro NISHIDA, Matsumoto (JP); Hideto ISHIGURO, Shiojiri (JP); Tsukasa EGUCHI, Matsumoto (JP)

(21) Appl. No.: 14/675,986

(22) Filed: Apr. 1, 2015

(30) Foreign Application Priority Data
Apr. 7, 2014 (JP) 2014-078417
Jul. 10, 2014 (JP) 2014-142022

(51) Int. Cl. A61B 5/1455; A61B 5/00; A61B 5/117; A61B 5/145

(52) U.S. Cl. CPC A61B 5/1455; A61B 5/14532; A61B 5/489; A61B 5/6826; A61B 5/7278; A61B 5/1172; A61B 2562/0238; A61B 2560/0252; A61B 2562/0242; A61B 2562/046

(57) ABSTRACT

A biological information acquisition apparatus includes a light source, a light-receiving unit, and a calculation unit. The light source is configured to irradiate a biological body with light. The light-receiving unit is configured to receive the light from the biological body. The calculation unit is configured to determine biological information. The calculation unit is configured to detect a first value by using a first signal from the light-receiving unit while the light source emits the light, detect a second value by using a second signal from the light-receiving unit while the light source does not emit the light, determine a temperature of the biological body based on the second value, and correct the first value using the temperature.

### CROSS-REFERENCE TO RELATED APPLICATIONS

This application claims priority to Japanese Patent Application No. 2014-078417 filed on Apr. 7, 2014 and Japanese Patent Application No. 2014-142022 filed on Jul. 10, 2014. The entire disclosures of Japanese Patent Application Nos. 2014-078417 and 2014-142022 are hereby incorporated herein by reference.

### BACKGROUND

1. Technical Field

The present invention relates to a biological information acquisition apparatus and a biological information acquisition method.

2. Related Art

A biological information acquisition apparatus is used, for example, in a biological authentication apparatus that uses captured image data such as the fingerprints, the veins, or the pupils of approved users in order to authenticate the approved users.

In addition, the biological information acquisition apparatus is used as a device that uses near-infrared light to perform noninvasive diagnostics on live bodies. For example, the biological information acquisition apparatus is used in blood glucose level measurements or analyses of biological components. In particular, spectroscopy that uses the absorption characteristics of matter is widely used.

The absorption characteristics of biological components are known to have large temperature dependence. In particular, the absorption characteristic of water has large temperature dependence. Therefore, in the biological information acquisition apparatus, a problem is the acquisition of accurate biological information because the absorption characteristics of light change when there are fluctuations in the temperature of the biological body, which is the measurement target.

Therefore, various technologies are studied to avoid the effects of temperature dependence of biological components. Japanese Laid-Open Patent Publication No. 2006-280762 discloses a biological measurement apparatus provided with a measurement means that measures the core body temperature in order to prevent the effects of temperature dependence of the biological components. Japanese Laid-Open Patent Publication No. 2010-227271 discloses a biological measurement apparatus that maintains a photodetection probe at nearly the same temperature as the core body temperature to avoid the effects of temperature dependence of the biological components.

In the conventional technology, however, because a means to measure the core body temperature and a holding means, and the like, were provided in the biological information acquisition apparatus, the biological information acquisition apparatus became large and heavy. Therefore, application was difficult in a biological information acquisition apparatus, for example, in the form of a wristwatch.

In addition, a problem was increased component costs because the configuration of the apparatus became complex.

### SUMMARY

An objective of the present invention is to propose a biological information acquisition apparatus and a biological information acquisition method that are able to avoid the effects of temperature dependence of the absorption characteristics of biological components without having to provide separate temperature measurement means and to acquire accurate biological information.

A biological information acquisition apparatus according to one aspect of the invention including a light source, a light-receiving unit, and a calculation unit. The light source is configured to irradiate a biological body with light. The light-receiving unit is configured to receive the light from the biological body. The calculation unit is configured to determine biological information. The calculation unit is configured to detect a first value by using a first signal from the light-receiving unit while the light source emits the light, detect a second value by using a second signal from the light-receiving unit while the light source does not emit the light, determine a temperature of the biological body based on the second value, and correct the first value using the temperature.

With the biological information acquisition apparatus according to one aspect of the invention, the light-receiving unit works as a first light-receiving element and as a second light-receiving element.

With the biological information acquisition apparatus according to one aspect of the invention, the second light-receiving element is arranged between the light source and the first light-receiving element.

With the biological information acquisition apparatus according to one aspect of the invention, the calculation unit is configured to detect the second value while the light-receiving unit is set in a light shielding state.

With the biological information acquisition apparatus according to one aspect of the invention, the biological information is at least one of a component concentration in the biological body, a component concentration in a blood and a blood glucose level.

With one aspect of the invention, a biological information acquisition method for receiving light irradiated from a light source toward a biological body at a light-receiving unit, and determining biological information includes detecting a first value by using the light-receiving unit while the light source emits light, detecting a second value by using the light-receiving unit while the light source does not emit the light, and calculating a temperature based on the second value, and correcting the first value using the temperature.

With the biological information acquisition method according to one aspect of the invention, the light-receiving unit works as a first light-receiving element and as a second light-receiving element.

With the biological information acquisition method according to one aspect of the invention, the biological information is at least one of a component concentration in the biological body, a component concentration in a blood, and a blood glucose level.

### BRIEF DESCRIPTION OF THE DRAWINGS

Referring now to the attached drawings which form a part of this original disclosure:

FIG. 1 is a diagram showing a biological information acquisition apparatus 1 related to an embodiment of the present invention;

FIG. 2 is a cross-sectional diagram of a biological information acquisition unit 12;

FIG. 3 is a schematic diagram showing an arrangement of lenses 44 in a light-focusing unit 40;

FIG. 4 is a circuit diagram of a light-receiving element 34; and

FIG. 5 is a flow chart showing a biological information acquisition method related to an embodiment of the present invention.

### DETAILED DESCRIPTION OF EXEMPLARY EMBODIMENTS

The biological information acquisition apparatus and the biological information acquisition method of an embodiment of the present invention are explained with reference to the drawings.

In order for the layers and the parts in the drawings to have sizes that enable recognition, the dimensions of the layers and the parts differ from the actual dimensions.

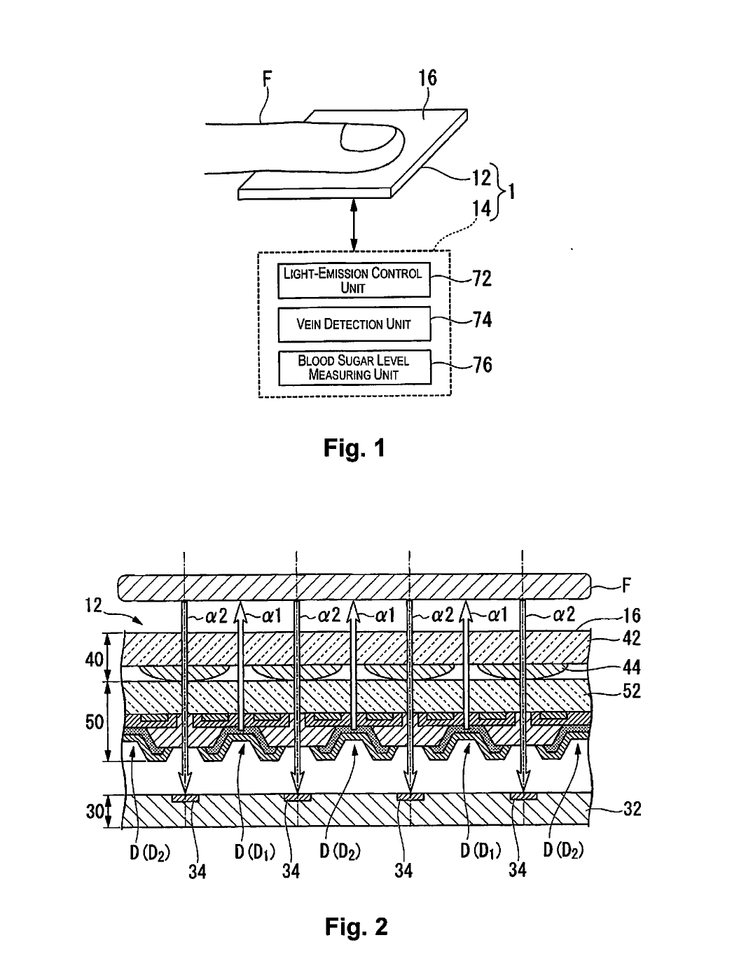

FIG. 1 is a diagram showing a biological information acquisition apparatus 1 related to one embodiment of the invention.

The biological information acquisition apparatus 1 detects such as a vein pattern of a finger F of the user, conducts personal authentication based on the vein pattern, and also measures the blood glucose level of the user.

The biological information acquisition apparatus 1 is provided with a biological information acquisition unit 12 and a control unit 14.

The biological information acquisition unit 12 acquires the vein pattern of the finger F. The finger F of the user is mounted on the front surface (detection surface 16) of the biological information acquisition unit 12.

The control unit 14 conducts personal authentication and health determination based on the information acquired by the biological information acquisition unit 12 (vein pattern or blood glucose level).

FIG. 2 is a cross-sectional diagram of the biological information acquisition unit 12.

The biological information acquisition unit 12 is configured to include a light-receiving unit 30, a light-focusing unit 40, and a light source unit 50.

The light-focusing unit 40 is arranged between the finger F and the light-receiving unit 30. The light source unit 50 is arranged between the light-receiving unit 30 and the light-focusing unit 40.

The light-receiving unit 30 is, for example, a complementary metal-oxide semiconductor (CMOS) sensor or a charge-coupled device (CCD) sensor. The light-receiving unit 30 is provided with a plate-shaped substrate 32 and a plurality of light-receiving elements 34. The plurality of the light-receiving elements 34 are formed on a surface at the finger F side (light-focusing unit 40 side) of the substrate 32 and arranged into an array form (matrix form). Each light-receiving element 34 generates and outputs a detection signal corresponding to the amount of received light. In this embodiment, as will be explained later, the light-receiving unit 30 is configured from light-receiving elements 34 in the form of CMOS sensors.

The light-focusing unit 40 is configured to include a substrate 42 and a plurality of lenses (microlenses) 44.

The substrate 42 is a light-transparent, plate-like part (e.g., glass substrate). The surface on the side opposite the light-receiving unit 30 side in the substrate 42 corresponds to the detection surface 16.

Each of the plurality of lenses 44 is formed on the surface on the light-receiving unit 30 side of the substrate 42 to have a one-to-one correspondence with each of the light-receiving elements 34 in the light-receiving unit 30. Each of the lenses 44 is a convex lens that focuses the incident light from the finger F side onto the light-receiving element 34 corresponding to that lens 44.

The substrate 42 and the plurality of lenses 44 can be formed as a unitary body.

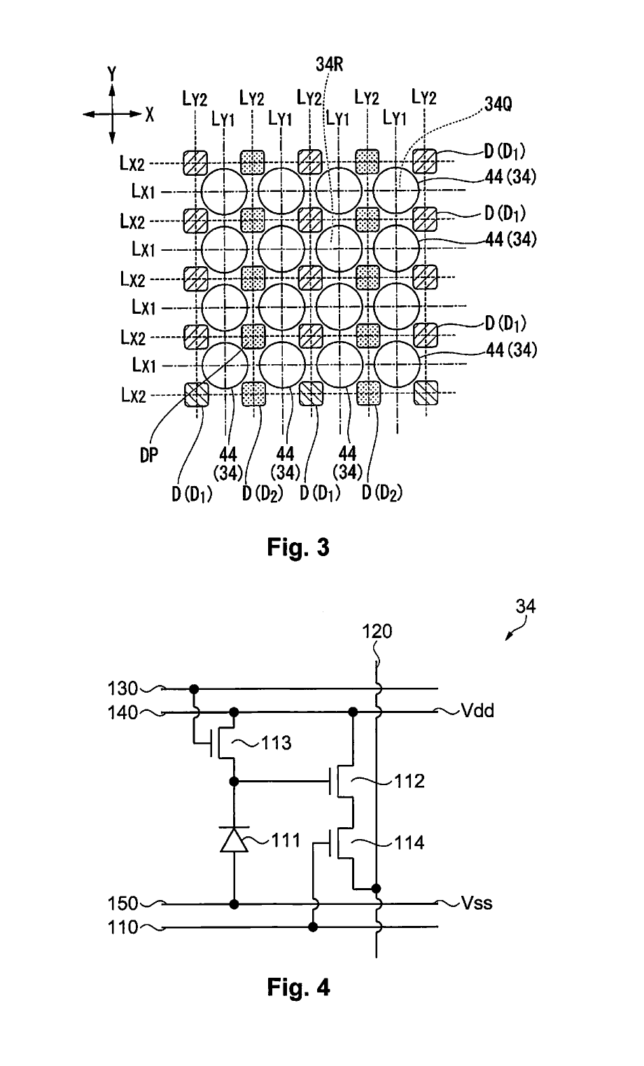

FIG. 3 is a schematic diagram showing the arrangement of each of the lenses 44 in the light-focusing unit 40.

The plurality of the lenses 44 are arranged into an array form along the X direction and the Y direction that are mutually orthogonal. Specifically, the lenses 44 are arranged so that the optical axis of each of the lenses 44 passes through an intersecting point of the plurality of straight lines LX1 extending in the X direction and the plurality of straight lines LY1 extending in the Y direction.

The optical axis of each lens 44 passes through the center of the light-receiving element 34 corresponding to that lens 44 (see FIG. 2).

As shown in FIG. 2, the light source unit 50 is configured to include a substrate 52 and a plurality of organic electroluminescent (EL) elements D (D1, D2).

The substrate 52 is a light-transparent, plate-like part (e.g., glass substrate).

The plurality of the organic EL elements D are thin-film light-emitting elements (light source) that irradiate the finger F with light having the specified wavelength (detection light). The plurality of the organic EL elements D are arranged in a matrix form along the X direction and the Y direction on the plane of the substrate 52.

As shown in FIG. 3, the plurality of the organic EL elements D are arranged at the intersecting parts of the plurality of straight lines LX2 and the plurality of straight lines LY2.

The plurality of the straight lines LX1 and the plurality of the straight lines LX2 are arranged alternately at equal intervals in the Y direction. The plurality of the straight lines LY1 and the plurality of the straight lines LY2 are arranged alternately at equal intervals in the X direction.

The plurality of the organic EL elements D have a first organic EL element D1 and a second organic EL element D2.

The first organic EL element D1 and the second organic EL element D2 irradiate detection light having mutually different wavelengths. The first organic EL element D1 emits detection light having wavelength λ1. The second organic EL element D2 emits detection light having wavelength λ2.

The wavelength λ1 and the wavelength λ2 are mutually different wavelengths in the wavelength range of near-infrared light. For example, wavelength λ1 is set to a numerical value that is absorbed by reduced hemoglobin in the veins of the finger F, and wavelength λ2 is set to a numerical value that is absorbed by glucose (grape sugar). A plurality of the first organic EL elements D1 are used to detect the vein pattern; and a plurality of the second organic EL elements D2 are used to measure the blood glucose level.

The arrangement of the first organic EL elements D1 positioned on the straight lines LY2 and the arrangement of the second organic EL elements D2 positioned on the straight lines LY2 are arranged to alternate at equal intervals in the X direction.

As indicated by arrow α1 in FIG. 2, the detection light emitted from each of the organic EL elements D passes through the substrate 52 and the substrate 42 of the light-focusing unit 40 and falls incident on the finger F. The light incident on the finger F is transmitted while being absorbed internally and is emitted from the finger F. Then, as indicated by arrow α2 in FIG. 2, light from the detection surface 16 falls incident on the light-focusing unit 40, is focused by each of the lenses 44, and arrives at the light-receiving elements 34.

The control unit 14 executes the detection of the vein pattern of the finger F and the measurement of the blood glucose level.

As shown in FIG. 1, the control unit 14 is configured to include a light-emission control unit 72, a vein detection unit 74, and a blood glucose level measuring unit 76. For example, a program stored in a memory circuit (not shown) is executed by a calculation processing apparatus (CPU) to implement each element of the control unit 14.

The light-emission control unit 72 controls each of the first organic EL elements D1 and each of the second organic EL elements D2 of the biological information acquisition unit 12 to selectively emit light. Specifically, the light-emission control unit 72 makes each of the first organic EL elements D1 irradiate detection light having wavelength λ1 when the vein pattern is detected and makes each of the second organic EL elements D2 irradiate detection light having wavelength λ2 when the blood glucose level is measured.

The vein detection unit (calculation unit) 74 detects the vein pattern of the finger F. The detection light having wavelength λ1 emitted by each of the first organic EL elements D1 reflects the vein pattern of the finger F in the amount of light received by each of the light-receiving elements 34 when wavelength λ1 is irradiated because the detection light is absorbed by the reduced hemoglobin in the vein.

The vein detection unit 74 uses the detection signals generated by the light-receiving elements 34 during the period in which the light-emission control unit 72 makes each of the first organic EL elements D1 irradiate detection light having wavelength λ1 to detect the vein pattern of the finger F. The vein detection unit 74 compares the vein patterns registered beforehand by legitimate users to the vein patterns specified by the actual detection signals. If the two patterns match, the user is judged to be legitimate (authentication success). If the two patterns do not match, the user is judged to not be legitimate (authentication failure).

The blood glucose level measuring unit (calculation unit) 76 measures the concentration of glucose (grape sugar) in the blood of the user. The detection light having wavelength λ2 that is detected by each of the second organic EL elements D2 is absorbed by glucose. Consequently, the concentration of glucose included in the user's blood is reflected in the amount of light received by each of the light-receiving elements 34.

The blood glucose level measuring unit 76 measures the blood glucose level corresponding to the detection signal that is generated by each of the light-receiving elements 34 within the period in which the light-emission control unit 72 makes each of the second organic EL element D2 irradiate the detection light at wavelength λ2.

The control unit 14 measures the concentration of glucose (grape sugar) by using the blood glucose level measuring unit 76 under the condition that authentication by the vein detection unit 74 was a success. The control unit 14 stores the result of measuring the glucose concentration (blood glucose level) by the blood glucose level measuring unit 76, and displays the blood glucose levels and the measurement history.

In addition, when authentication by the vein detection unit 74 failed, measuring the concentration of glucose by the blood glucose level measuring unit 76 is halted.

FIG. 4 is a circuit diagram of the light-receiving element 34. The anode of a photodiode 111 is connected to a negative power supply line 150. A negative power supply voltage Vss is supplied to the negative power supply line 150. The gate of a gain transistor 112 and the source of a reset transistor 113 are connected to the cathode of the photodiode 111. The drain of the gain transistor 112 and the drain of the reset transistor 113 are connected to a positive power supply line 140, and a positive power supply voltage Vdd is supplied to the positive power supply line 140. The source of the gain transistor 112 is connected to the drain of a select transistor 114. The source of the select transistor 114 is connected to a read line 120, and the gate of the select transistor 114 is connected to a scan line 110. And, the gate of the reset transistor 113 is connected to the reset signal line 130.

When the light-receiving element 34 measures the amount of light, the gate of the gain transistor 112 is initially charged to the positive power supply voltage Vdd. Next, light is exposed over a period of τ. The reset transistor 113 during the light exposure period is set in the off state, which causes the gate voltage Vg of the gain transistor 112 to change in response to the coupled leakage current I of the photodiode 111. After the light exposure ends, the gate voltage of the gain transistor 112 becomes Vg=Vdd−Iτ/C_{T}. Here, C_{T} is the transistor capacitance of the gain transistor 112. The coupled leakage current increases as the amount of light increases, and the gate voltage Vg of the gain transistor 112 changes in response to the amount of light. As a result, the changes in the generated conductance of the gain transistor 112 are measured at each light-receiving element 34 during the read-out period, and the amount of light irradiated during the light exposure period is measured.

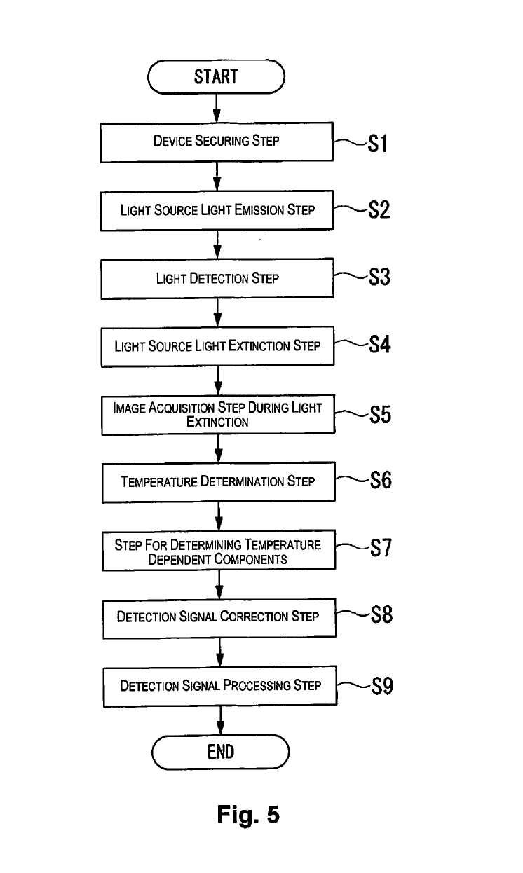

FIG. 5 is a flow chart showing the biological information acquisition method related to the embodiment of the invention.

The blood (hemoglobin or glucose) has large temperature dependence. In particular, the absorption characteristics of water have large temperature dependence.

Therefore, the acquisition of accurate biological information is difficult because the light absorption characteristics change when the body temperature of the user fluctuates. That is, the detection of the vein pattern or the measurement of the blood glucose level becomes incorrect.

Therefore, the biological information acquisition apparatus 1 references the output value of the light-receiving unit 30 (light-receiving element 34) during light extinction, and measures the body temperature of the user (biological body). Then, the detection result of the vein pattern or the blood glucose level is corrected based on the measured body temperature. By doing this, the detection of the vein pattern and the measurement of the blood glucose level are accurately performed. That is, accurate biological information is acquired.

Through diligent research, the inventors of the application discovered that the photodiode 111 exhibits strong temperature dependence. A PN junction semiconductor diode in the reverse bias state is used as the photodiode 111. The generation principle of the PN junction leakage current is the generation of electron-hole pairs in the depleted region by Shockley-Reed-Hall generation or the phonon-assisted tunneling that accompanies the Pool-Frankel effect. In this case, a minute change in temperature changes the spread of the Fermi function. Therefore, the frequency of Shockley-Reed-Hall generation or the frequency of phonon-assisted tunneling accompanying the Pool-Frankel effect varies greatly. Therefore, the photodiode 111 exhibits strong temperature dependence. Consequently, the measurement current during light extinction in which light does not fall incident on the photodiode 111 contains temperature information.

Specifically, the biological information acquisition method is conducted in the following steps. More specifically, the following steps S2-S9 are performed under the control of the control unit 14.

First, the biological information acquisition apparatus 1 is attached to the user's finger F. Namely, the detection surface 16 is tightly affixed to the finger F (device securing step S1).

Next, among the plurality of organic EL elements D, an arbitrary organic EL element (first light source) DP is controlled to emit light (light source emission step S2). The organic EL element DP may be either the first organic EL element D1 or the second organic EL element D2.

In the state in which the organic EL element DP emits light, the light randomly transmitted in the interior of the biological body (finger F) is received (detected) by the light-receiving element (first light-receiving element 34Q) positioned apart from the organic EL element DP (light detection step (detection step during light emission) S3). In other words, the detection signal of the first light-receiving element 34Q (detection value during light emission) is acquired.

The light emitted from the organic EL element DP is absorbed by at least one of the hemoglobin and glucose included in the blood, and at least one of vein pattern information of the finger F and blood glucose level information is included in the light received (detected) by the first light-receiving element 34Q.

When light reception by the first light-receiving element 34Q is completed, the organic EL element DP is controlled to stop emitting of the light (light source light extinction step S4).

Next, in the state in which the organic EL element DP does not emit light, the detection signal of the second light-receiving element 34R (detection value during light extinction) is acquired. A light-receiving element 34 works as the first light-receiving element 34Q while the organic EL elements D emit light, and works as the second light-receiving element 34R while the organic EL elements D do not emit light. Because the second light-receiving element 34R does not detect light, the second light-receiving element 34R primarily outputs currents that reflect the temperature information to the read lines 120. Namely, the second light-receiving element 34R acquires an image when the light source does not emit light (image acquisition step during light extinction (detection step during light extinction) S5).

There is temperature dependence in the image during light extinction that is acquired by the second light-receiving element 34R. Therefore, temperature information of the finger F (biological body) is included in the image during light extinction acquired by the second light-receiving element 34R.

Next, based on the image during light extinction acquired by the second light-receiving element 34R during light extinction, the temperature of the finger F (biological body) is determined (temperature determination step S6). This time, a conversion table that was prepared in advance is referenced.

Namely, the relationship (temperature dependence) between the temperature and the current values (referred to as the detection value during light extinction) from the second light-receiving element 34R that become the image during light extinction is verified (examined) in advance. A conversion table that determines the temperature from the detection value during light extinction of the second light-receiving element 34R is created.

Consequently, by comparing the detection value during light extinction from the second light-receiving element 34R to the conversion table, the temperature of the finger F (biological body) can be determined. Preferably, the detection value during light extinction are detected when the second light-receiving element 34R is in the light shielding state. Thus, the detection value during light extinction, when external light is blocked, the light source does not emit light, and light is shielded, accurately reflects the temperature information.

Next, based on the temperature determined in the temperature determination step S6, the temperature dependent components included in the detection signal of the first light-receiving element 34Q are determined (step for determining temperature dependent components S7). This time, a conversion table that was prepared in advance is referenced.

Namely, the relationship between the detection signal of the first light-receiving element 34Q and the body temperature (temperature dependent component) is verified (studied) in advance. A conversion table that determines the temperature dependent components included in the detection signal of the first light-receiving element 34Q from the body temperature is created.

Consequently, by comparing the body temperature to the conversion table, the temperature dependent components included in the detection signal of the first light-receiving element 34Q can be determined.

Next, the detection signal acquired in light detection step S3 is corrected (detection signal correction step (correction step) S8). Namely, the temperature dependent components determined in the step for determining temperature dependent components S7 are removed from the detection signal acquired in light detection step S3. Thus, a detection signal with the effects of the body temperature removed is determined.

Finally, the detection signal obtained after passing through detection signal correction step S8 is calculated (detection signal processing step S9). In detection signal processing step S9, for example, multivariate analysis is conducted.

By doing this, the vein pattern or the blood glucose level of the biological body (finger F) is obtained. The effects of the body temperature are removed from the vein pattern and the blood glucose level.

When the blood glucose level is determined in the detection signal processing step S9, for example, the calculation processes shown below are conducted.

When the main components in a biological body are considered to be water, protein, lipid, and glucose, equation (1) is established from the Lambert-Beer law as follow:

$$\{ \begin{matrix}A ( {λ}_{1} ) = {ɛ}_{w} ( {λ}_{1} ) {c}_{w} L + {ɛ}_{p} ( {λ}_{1} ) {c}_{p} L + {ɛ}_{l} ( {λ}_{1} ) {c}_{l} L + {ɛ}_{g} ( {λ}_{1} ) {c}_{g} L \\ A ( {λ}_{2} ) = {ɛ}_{w} ( {λ}_{2} ) {c}_{w} L + {ɛ}_{p} ( {λ}_{2} ) {c}_{p} L + {ɛ}_{l} ( {λ}_{2} ) {c}_{l} L + {ɛ}_{g} ( {λ}_{2} ) {c}_{g} L \\ A ( {λ}_{3} ) = {ɛ}_{w} ( {λ}_{3} ) {c}_{w} L + {ɛ}_{p} ( {λ}_{3} ) {c}_{p} L + {ɛ}_{l} ( {λ}_{3} ) {c}_{l} L + {ɛ}_{g} ( {λ}_{3} ) {c}_{g} L \\ A ( {λ}_{4} ) = {ɛ}_{w} ( {λ}_{4} ) {c}_{w} L + {ɛ}_{p} ( {λ}_{4} ) {c}_{p} L + {ɛ}_{l} ( {λ}_{4} ) {c}_{l} L + {ɛ}_{g} ( {λ}_{4} ) {c}_{g} L\end{matrix} \qquad ( 1 )$$

where A is absorbance, L is optical path length (constant regardless of wavelength), ε_{w}, εₚ, εₗ, ε_{g} are molar light absorption coefficients of water, protein, lipid, glucose, and c_{w}, cₚ, cₗ, C_{g} are molar concentrations of water, protein, lipid, glucose.

When four wavelengths are used to acquire the absorbances A (detection signals), then the cL values, which are the products of the respective concentration c of a main component in the biological body multiplied by the optical path length L, are determined from equation (1).

Namely, c_{w}L, cₚL, cₗL, C_{g}L are determined.

Consequently, if the optical path length L is known, the respective concentration c of the main components (water, protein, lipid, glucose) in the biological body can be calculated.

For example, the respective concentration of the main components in the biological body can be determined in advance by sampling the blood. Thus, accurate biological information with the effects of the body temperature removed can be obtained by the biological information acquisition method used by the biological information acquisition apparatus 1.

The biological information acquisition apparatus 1 is not limited to the embodiment described above, and the same effects of the embodiment are obtained even in modes such as the modified examples given next.

The light-receiving element 34Q and the light-receiving element 34R may be the same light-receiving element. That is, light-receiving element 34R may also be light-receiving element 34Q.

The biological information acquisition unit 12 is not limited to acquiring two types of biological information (vein pattern, blood glucose level). The acquisition may be the biological information of only either one of the vein pattern or the blood glucose level.

The biological information may be brain waves, myoelectricity, cardioelectricity, pulse rate (pulse), blood pressure, and the like.

A biological information acquisition apparatus related to one aspect of the embodiment including a light source, a light-receiving unit, and a calculation unit. The light source is configured to irradiate a biological body with light. The light-receiving unit is configured to receive the light from the biological body. The calculation unit is configured to determine biological information based on the light received at the light-receiving unit. The calculation unit is configured to detect a detection value during light emission by using the light-receiving unit while the light source emits the light, detect a detection value during light extinction by using the light-receiving unit while the light source does not emit the light, determine a temperature dependent component of the detection value during the light emission based on the detection value during the light extinction, and correct the detection value during the light emission using the temperature dependent component.

Consequently, the embodiment is able to acquire high-precision biological information with the effects of temperature dependence of the biological body components removed from the detection value during light emission. Additionally, in the embodiment, because the information related to the temperature of the biological body is also acquired from the light-receiving elements, devices for acquiring temperature information do not have to be separately provided which can contribute to a smaller size, a lighter weight, and a lower cost of the apparatus.

With the biological information acquisition apparatus related to a second aspect of the embodiment, the light-receiving unit includes a first light-receiving element and a second light-receiving element.

Thus, in the embodiment, the detection value during light emission can be acquired by the first light-receiving element, and the detection value during light extinction can be acquired by the second light-receiving element.

With the biological information acquisition apparatus related to a third aspect of the embodiment, the second light-receiving element is arranged between the light source and the first light-receiving element.

By doing this, in the embodiment, even when the detection value during light extinction is slightly weaker than the detection value during light emission, the second light-receiving element that is able to precisely acquire the detection value during light extinction can be selected.

With the biological information acquisition apparatus related to a fourth aspect of the embodiment, the calculation unit is configured to detect the detection value during the light extinction with the light-receiving unit set in a light shielding state.

By doing this, in the embodiment, the detection value during light extinction, when the light source is not emitting light and light is shielded, accurately reflects the temperature information. By understanding the relationship between the detection value during light extinction and the temperature of the biological body, the body temperature can be easily acquired.

With the biological information acquisition apparatus related to a fifth aspect of the embodiment, the biological information is at least one of a component concentration and a blood glucose level in at least one of a biological body and a blood.

By doing this, in the embodiment, information related to the component concentration or the blood glucose level in the body or in the blood that have large temperature dependence can be acquired with high precision.

A biological information acquisition method related to a sixth aspect of the embodiment is a biological information acquisition method in which the light irradiated from a light source toward a biological body is received by a light-receiving elements to determine biological information, and includes a detection step during light emission in which the light source emits the light, and a detection value during light emission is detected by the light-receiving unit, a detection step during light extinction in which the light source does not emit the light, and the detection value during light extinction is detected by the light-receiving unit, and a correction step in which a temperature dependent component of the detection value during the light emission is determined based on the detection value during the light extinction, and the detection value during light emission is corrected.

Consequently, in the embodiment, high-precision biological information from which the effects of temperature dependence of the biological body components were removed from the detection value during light emission can be acquired. In addition, in the embodiment, because information related to the temperature of the biological body is also acquired by the light-receiving element, a device for acquiring temperature information does not have to be provided separately which can contribute to the smaller size, the lighter weight, and the lower cost of the apparatus.

With the biological information acquisition method related to the seventh aspect of the embodiment, the light-receiving unit includes a first light-receiving element and a second light-receiving element.

By doing this, in the embodiment, for example, the detection value during light emission can be acquired by the first light-receiving element, and the detection value during light extinction can be acquired by the second light-receiving element.

With the biological information acquisition method related to the eighth aspect of the embodiment, the biological information is at least one of a component concentration and a blood glucose level in at least one of a biological body and a blood.

Thus, in the embodiment, information related to the component concentration or the blood glucose level in the biological body or in the blood that has large temperature dependence can be acquired with high precision.

### GENERAL INTERPRETATION OF TERMS

In understanding the scope of the present invention, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” or “element” when used in the singular can have the dual meaning of a single part or a plurality of parts. Finally, terms of degree such as “substantially”, “about” and “approximately” as used herein mean a reasonable amount of deviation of the modified term such that the end result is not significantly changed. For example, these terms can be construed as including a deviation of at least ±5% of the modified term if this deviation would not negate the meaning of the word it modifies.

While only selected embodiments have been chosen to illustrate the present invention, it will be apparent to those skilled in the art from this disclosure that various changes and modifications can be made herein without departing from the scope of the invention as defined in the appended claims. Furthermore, the foregoing descriptions of the embodiments according to the present invention are provided for illustration only, and not for the purpose of limiting the invention as defined by the appended claims and their equivalents.

What is claimed is:

1. A biological information acquisition apparatus comprising:
a light source configured to irradiate a biological body with light;
a light-receiving unit configured to receive the light from the biological body; and
a calculation unit configured to determine biological information,
the calculation unit being configured to detect a first value by using a first signal from the light-receiving unit while the light source emits the light, detect a second value by using a second signal from the light-receiving unit while the light source does not emit the light, determine a temperature of the biological body based on the second value, and correct the first value using the temperature.

2. The biological information acquisition apparatus according to claim 1, wherein
the light-receiving unit works as a first light-receiving element and as a second light-receiving element.

3. The biological information acquisition apparatus according to claim 2, wherein
the second light-receiving element is arranged between the light source and the first light-receiving element.

4. The biological information acquisition apparatus according to claim 1, wherein
the calculation unit is configured to detect the second value while the second light-receiving unit is set in a light shielding state.

5. The biological information acquisition apparatus according to claim 1, wherein
the biological information is at least one of a component concentration in the biological body, a component concentration in a blood and a blood glucose level.

6. A biological information acquisition method for receiving light irradiated from a light source toward a biological body at a light-receiving unit, and determining biological information, the biological information acquisition method comprising:
detecting a first value by using the light-receiving unit while the light source emits light;
detecting a second value by using the light-receiving unit while the light source does not emit the light; and
calculating a temperature based on the second value, and correcting the first value using the temperature.

7. The biological information acquisition method according to claim 6, wherein
the light-receiving unit works as a first light-receiving element and as a second light-receiving element.

8. The biological information acquisition method according to claim 6, wherein
the biological information is at least one of a component concentration in the biological body, a component concentration in a blood, and a blood glucose level.
